(19) United States
(12) Patent Application Publication
(10) Pub. No.: US 2014/0073987 A1
(43) Pub. Date: Mar. 13, 2014

(54) MINIMALLY INVASIVE SKIN BIOPSY DEVICE FOR REMOVAL OF SKIN CONDITIONS IN THE EPIDERMIS

(71) Applicant: THE JOHNS HOPKINS UNIVERSITY, Baltimore, MD (US)

(72) Inventors: Sewon Kang, Phoenix, MD (US); Danielle Nicole Dorfman, Miami, FL (US); Yifu Ding, Souderton, PA (US); Chirag Shah, San Jose, CA (US); Andrew S. Harwell, Baltimore, MD (US); Marie Kelly, Baltimore, MD (US); Anna Chien, Columbia, MD (US); Luis Garza, Baltimore, MD (US); Janice Taube, Baltimore, MD (US); Anish Bhatnagar, Sandy, UT (US); Desai Pujan, Houston, TX (US)

(73) Assignee: THE JOHNS HOPKINS UNIVERSITY, Baltimore, MD (US)

(21) Appl. No.: 14/116,456

(22) PCT Filed: May 9, 2011
(86) PCT No.: PCT/US2012/037005

§ 371 Date: Nov. 8, 2013

Related U.S. Application Data
(60) Provisional application No. 61/483,926, filed on May 9, 2011.

(51) Int. Cl. A61B 10/02

(52) U.S. Cl. CPC A61B 10/0266
USPC 600/564

(57) ABSTRACT

An embodiment in accordance with the present invention provides a device for performing a skin biopsy that includes a generally curved blade to cut the skin at a predetermined depth disposed within or an integral part of a blade holder assembly. The blade holder assembly has an outer surface and the generally curved blade is coupled to the outer surface of the blade holder assembly. The device also includes a housing having an outer wall and said outer wall defining an interior space within the housing, such that the blade holder assembly and generally curved blade are disposed substantially within the inner space of the housing. The device also includes a handle coupled to the blade holder assembly and configured to rotate the blade holder assembly and in turn the generally curved blade within the inner space of the housing to perform the skin biopsy or removal of skin conditions.

### CROSS REFERENCE TO RELATED APPLICATION

This application claims the benefit of U.S. Provisional Patent Application No. 61/483,926 filed May 9, 2011, which is incorporated by reference herein, in its entirety.

### FIELD OF THE INVENTION

The present invention relates generally to skin treatment. More particularly, the present invention relates to a device for minimally invasive skin biopsy and removal of skin conditions.

### BACKGROUND OF THE INVENTION

There are many skin conditions and cancers that are specific to the epidermis including, but not limited to, seborrheic keratosis, acinic keratosis, and basal cell carcinoma. The current standard of care is a curved razor blade, that is used to shave what the doctor presumes is the epidermal layer of the skin lesion. This reduces contact with nerve endings and blood vessels found in the dermal layer of the skin. Performing the procedure with a razor blade can expose a physician to razor pricks and blood-borne infection and also increase the difficulty of the procedure, as there is no guide for the depth of the cut.

It would therefore be advantageous to provide a device to control the depth of the cut as well as protect he physician from the blade of the device.

### SUMMARY OF THE INVENTION

The foregoing needs are met, to a great extent, by the present invention, wherein in one aspect a device for performing a biopsy of skin includes a generally curved blade configured to cut the skin at a predetermined depth. The device also includes a blade holder assembly configured such that the generally curved blade is coupled to the blade holder assembly. A housing of the device includes an outer wall defining an interior space within the housing, such that the blade holder assembly and generally curved blade are disposed within the inner space of the housing. The device also includes a handle coupled to the blade holder assembly and configured to role the blade holder assembly, and in turn, the generally curved blade, within the inner space of the housing.

In accordance with another aspect of the present invention the blade holder assembly is generally spherical in shape and includes an outerhousing defining a hollow interior space. The blade holder assembly can be formed from a material such as a biocompatible metal or plastic. The housing is generally cylindrical shape and can be formed from a generally clear material. A height of a position of the generally spherical blade holder within the housing correlates to a depth of a cut of the curved blade into the skin. For instance, the depth of the cut of the curved blade is configured to be at least 0.2 mm. The handle further can include an outer wall defining a hollow interior space within the handle.

In accordance with still another aspect of the present invention, a device for performing a biopsy of skin includes a generally spherical blade assembly having an outer wall defining a hollow interior space, and wherein the outer wall further defines an opening into the hollow interior space, The device also includes a blade wherein the blade is an edge of the outer wall of the generally spherical blade assembly adjacent to the opening into the hollow interior space and wherein an edge of the blade is sharpened. Additionally, the device includes a housing having an outer wall. The outer wall defines an interior space within the housing, such that the generally spherical blade assembly is disposed substantially within the inner space of the housing. A handle is coupled to the generally spherical blade assembly and configured to rotate the generally spherical blade assembly within the inner space of the housing.

In accordance with yet another aspect of the present invention, the opening into the hollow interior space of the generally spherical blade assembly has a wedge shape, and the generally spherical blade assembly can be formed from a metal or a plastic. The housing is generally cylindrical in shape and can be formed from a generally clear material. A height of a position of the generally spherical blade holder within the housing correlates to a depth of a cut of the curved blade into the skin. The depth of the cut of the blade can be configured to be at least 0.2 mm. The handle further includes an outer all defining a hollow interior space.

In accordance with even another aspect of the present invention, a method of performing a skin biopsy includes positioning a housing for a device to perform skin biopsy over an area of skin to be biopsied. The method also includes rotating a generally spherical blade assembly within the housing and cutting the area of skin to be biopsied with a blade disposed in the generally spherical blade assembly to create a skin sample for biopsy. The skin sample can be contained for biopsy within the generally spherical blade assembly,

In accordance with yet another aspect of the present invention, the method can further include controlling a depth of a cut of the blade into the skin by adjusting to a height of a position of the generally spherical blade assembly within the housing. The depth can be configured to be at least 0.2 mm. In addition, the method can include rotating the generally spherical blade assembly with a handle coupled to the generally spherical blade assembly.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIG. 1 illustrates a side view of a skin biopsy device in a starting position according to an embodiment of the present invention.

FIG. 2 illustrates a side view of a skin biopsy device in a rotated position according to the embodiment of the invention illustrated in FIG. 1.

FIG. 3 illustrates a perspective view of a spherical blade holder and blade according to an embodiment of the present invention.

FIG. 4 illustrates a sectional view of the spherical blade holder and blade according to the embodiment illustrated in FIG. 3.

FIG. 5 illustrates a perspective view of a housing according to an embodiment of the present invention.

FIG. 6 illustrates a first sectional view of the housing according to the embodiment of the present invention illustrated in FIG. 5.

FIG. 7 illustrates a second sectional view of the housing according to the embodiment of the present invention illustrated in FIGS. 5 and 6.

FIG. 8 illustrates a perspective view of a handle according to an embodiment of the present invention.

FIG. 9 illustrates a sectional view of the handle according to the embodiment of the invention illustrated in FIG. 8.

### DETAILED DESCRIPTION

The presently disclosed subject matter now will be describe hereinafter with reference to the accompanying Drawings, in which some, but not all embodiments of the inventions are shown. Like numbers refer to like elements throughout, The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides a device for performing a skin biopsy that includes a generally curved blade to cut the skin at a predetermined depth disposed within or an integral part of a blade holder assembly. The blade holder assembly has an outer surface and the generally curved blade is coupled to the outer surface of the blade holder assembly. The device also includes a housing having an outer wall and said outer wall defining an interior space within the housing, such that the blade holder assembly and generally curved blade are disposed substantially within the inner space of the housing. The device also includes a handle coupled to the blade holder assembly and configured to rotate the blade holder assembly and in turn the generally curved blade within the inner space of the housing to perform the skin biopsy or removal of other skin conditions.

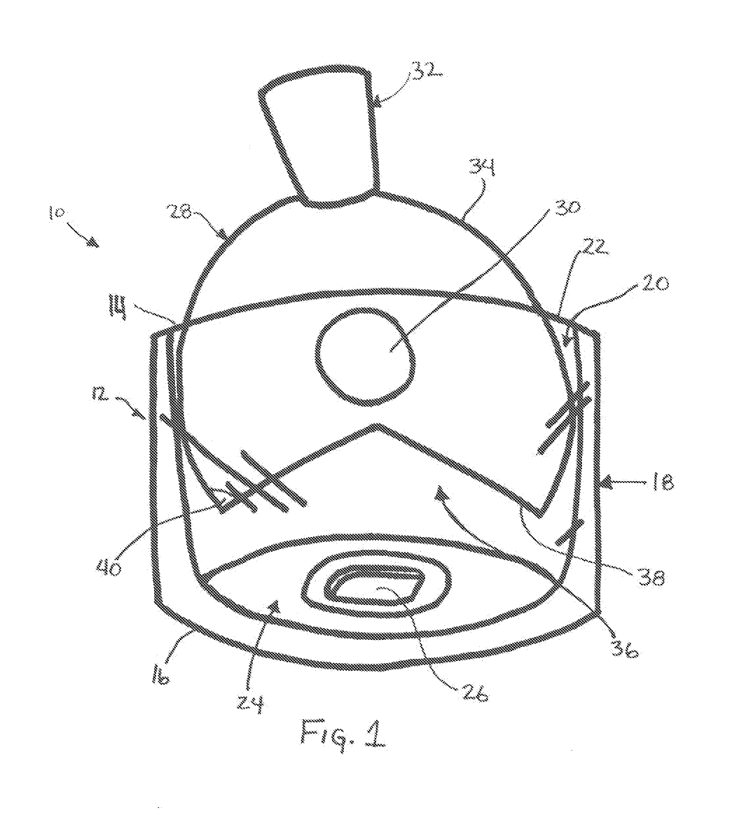

FIG. 1 illustrates a side view of a skin biopsy device in a starting position according to an embodiment of the present invention. As illustrated in FIG. 1, the skin biopsy device 10 includes an outer housing 12. The outer housing 12 is illustrated as being generally cylindrical in shape, The housing 12 has a top edge 14 and a bottom edge 16 and also includes a side wall 18 defining a hollow interior space 20. The side wall 18 defines un opening 22 at the top edge 14 of the housing 12. Additionally, the outer housing 12 also includes bottom wall 24, positioned near a bottom edge 16 of the housing 12. The bottom wall 24 defines a sample hole 26, positioned approximately at a middle point of the bottom wall 24 of the housing 12. The housing 12 is shown in FIG. 1 as being formed from a transparent material, in order to enhance physician visualization of a skin biopsy procedure. However, any suitable material known to one of skill in the art can be used to form the housing 12.

The skin biopsy device 10, as illustrated in FIG. 1, also includes a blade holder assembly 28 disposed within the hollow interior space 20 of the housing 12. The blade holder assembly 28 is illustrated as being generally spherical in shape, but can be any other shape suitable for performing a skin biopsy procedure known to one of skill in the art. The blade holder assembly 28 is coupled to the housing 12 with a bolt or pin 30. Preferably, the blade holder assembly pivots about the bolt or pin 30. The blade holder assembly 28 has a handle 32 coupled to an outer wall 34 of the blade holder assembly 28. As illustrated in FIG. 1, the outer wall 34 of the blade holder assembly 28 defines an opening 36 haying an outer edge 38, such that a blade 40 can be positioned along at least a portion of the outer edge 38 of the opening 36.

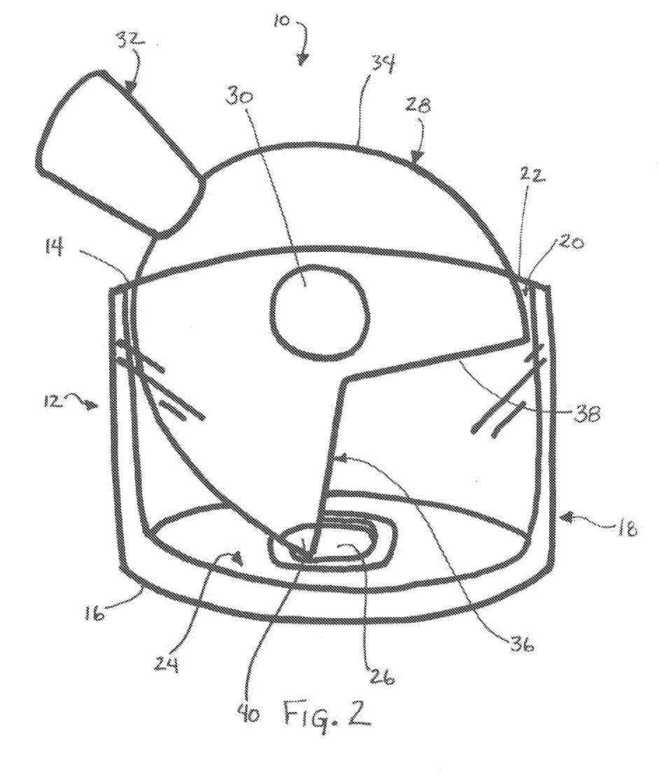

FIG. 2 illustrates a side view of a skin biopsy device in a rotated position according to the embodiment of the invention illustrated in FIG. 1. Both the housing 12 and the blade holder assembly 28 are configured such that the blade holder assembly 28 is rotatable, such as about bolt or pin 30, within the hollow interior space 20 of the housing 12. FIG. 2 illustrates this rotation of the blade holder assembly 28 within the hollow interior space 20 of the housing 12, from the starting position illustrated in FIG. 1, to the rotated position illustrated in FIG. 2. As illustrated herein, the blade holder assembly 28 rotates about only one axis, such as a transverse axis of the bolt or pin 30. However, the blade holder assembly 28 could also be coupled to the housing 12 in such a way that the blade holder assembly 28 pivots or rotates about multiple axes. FIG. 2 shows that in the rotated position the blade is configured to penetrate the skin by a predetermined depth. The predetermined depth of penetration into the skin is at least 0.2 mm and is preferably 0.3 mm. This depth allows for a sufficient biopsy of the patient's skin without penetrating the patient's dermal layer. As the dermal layer is filled with nerve endings and blood vessels, this can prevent excess bleeding and nerve damage. However, the depth of penetration can be adjusted by adjusting a height at which the blade holder assembly 28 sits within the hollow interior space 20 of the housing 12. The height at which the blade assembly sits within the hollow interior space 20 of the housing 12 can be changed by adjusting the height at which pin or bolt 30 is coupled to housing 12 or the height at which the pin or bolt 30 is coupled to the blade assembly 28. If the height of the blade holder assembly 28 is raised higher up above the top edge 14 of the housing 12 the depth of the cut will be lesser and if the height of the blade holder assembly is lowered below the top edge 14 of the housing 12 the depth of the cut will be greater. Any depth of skin penetration known to one of reasonable skill in the art could be used with respect to this invention.

Further, as illustrated in FIG. 2 the hole 26 defined by the bottom wall 24 of the housing 12 allows the blade 40 to penetrate the skin for biopsy. Therefore, the device 10 is preferably positioned on the patient's skin, such that the skin to be biopsied is accessible via hole 26. The bottom wall 24 can also act as a safety mechanism for preventing the blade 40 from cutting too deep into the skin or cutting too large of a biopsy region.

The method of using the device can also be described with respect to FIG. 2, although these steps are not meant to limit the potential ways in which one of skill in the art could envision using the device. The method includes positioning the housing 12 of the device 10 over an area of skin to be biopsied. The blade holder assembly 28 is rotated within the housing 12 of the device 10. As the blade holder assembly 28 is rotated, the blade 40 begins to cut a sample of the area of skin to be biopsied. This skin sample is contained for biopsy within the blade assembly 28. Alternately, the skin sample can be transferred to any biopsy sample container known to one of skill in the art. As noted above, the depth of the cut of the blade 40 into the skin can be adjusted by changing the height of the position of the blade assembly 28 within the housing 12. Preferably the depth of the cut is approximately 0.3 mm, but can be any other depth known to one of skill in the art, such as at least 0.2 mm Additionally, the blade assembly 28 can be rotated with the handle 32 coupled to the blade assembly 28.

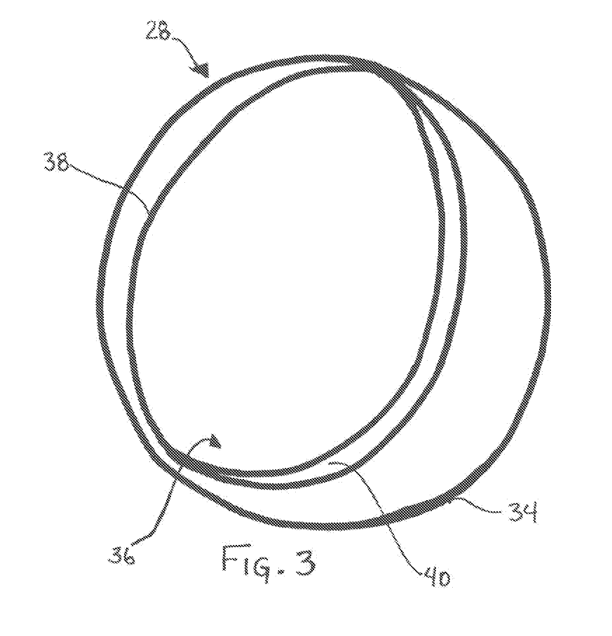

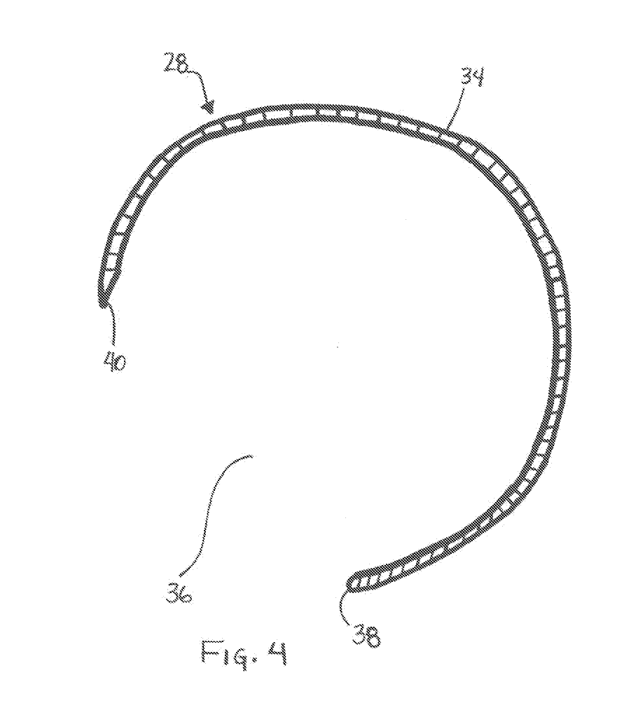

FIG. 3 illustrates a perspective view of the spherical blade holder assembly and blade according to an embodiment of the present invention and FIG. 4 illustrates a sectional view of the spherical blade holder and blade according to the embodiment illustrated in FIG. 3. As illustrated in FIGS. 3 and 4, the blade holder 28 is illustrated as being generally spherical in shape, but can be any other shape suitable for performing a skin biopsy procedure known to one of skill in the art. The blade holder assembly 28 has the outer wall 34 of the blade holder assembly 28, which defines the generally wedge shaped opening 36 having an outer edge 38, such that the blade 40 can be positioned along at least a portion of the outer edge 38 of the opening 36. As illustrated in the sectional view of FIG. 4, the opening 36 preferably has a wedge shape defined by the outer wall 34 of the blade assembly 28. The outer wall 34 also preferably defines an at least partially hollow inner space 40. The blade 40 can be manufactured integrally with or separately from the blade holder assembly 28. If the blade 40 is to be manufactured integrally with the blade holder assembly, it is preferable that the assembly 28 and the blade 40 are formed from a biocompatible metal, such as a stainless steel. At least a portion of the outer edge 38 of the opening 36 can then be machined to form a sharp edge for the blade, Alternately, the assembly 28 can be manufactured from a plastic material and the blade 40 can be injection molded into the assembly using a biocompatible metal material. Another way to vary the depth of the cut into the skin, is to vary the dimensions of the blade holder assembly 28 or alternately, the angle of the blade 40 with respect to the outer edge 38 of the assembly 28.

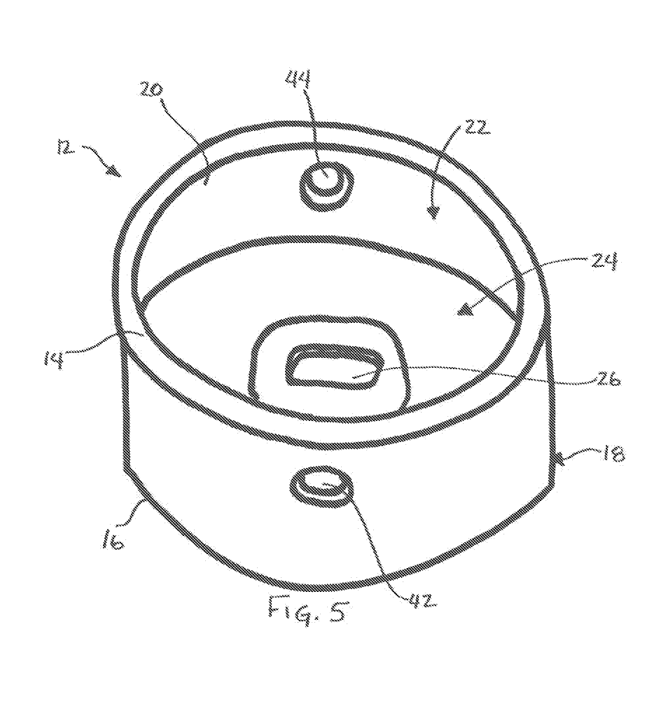

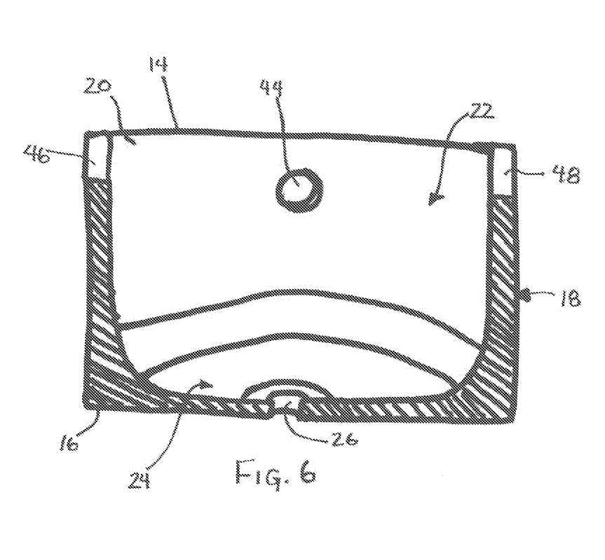

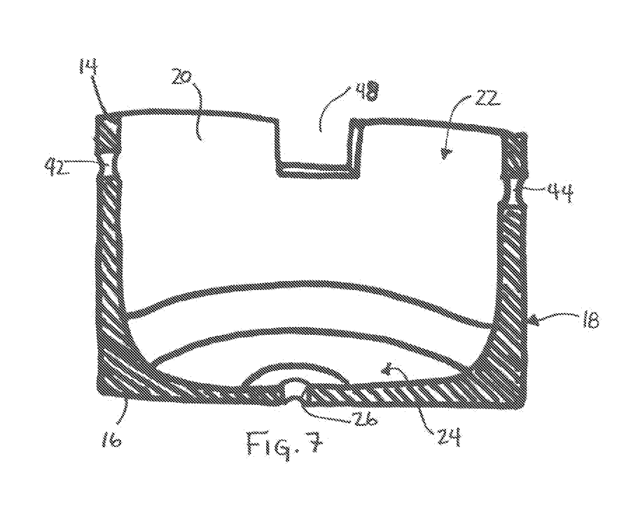

FIG. 5 illustrates a perspective view of a housing and FIG. 6 illustrates a first sectional view of the housing according to he embodiment of the present invention illustrated in FIG. 5. FIG. 7 illustrates a second sectional view of the housing according to the embodiment of the present invention illustrated in FIGS. 5 and 6. As discussed above and also here, with respect to FIGS. 5, 6, and 7, the housing 12 is generally cylindrical in shape. The housing 12 has top edge 14 and bottom edge 16 and also includes side wall 18 defining hollow interior space 20. The side wall 18 defines opening 22 at the top edge 14 of the housing 12.

Additionally, the outer housing 12 also includes bottom wall 24, positioned near bottom edge 16 of the housing 12. The bottom wall 24 defines sample hole 26, positioned approximately at a middle point of the bottom wall 24 of the housing 12. Sample hole 26 is configured such that the blade can be used to take a sample of the skin to be biopsied. The size of sample hole 26 can be changed based on the type of skin biopsy to be performed. Alternately, the device can also be configured such that a physician can increase or decrease the size of the hole 26. For instance, bottom wall 24 can define a reduction in thickness surrounding sample hole 26 such that a preferred depth of skin sample can be taken with the biopsy device. The housing 12 is formed from a transparent material, in order to enhance physician visualization of a skin biopsy procedure. However, any suitable material known to one of skill in the art can be used to form the housing 12.

In addition, as illustrated in FIGS. 5, 6, and 7, the housing 12 defines pin holes 42 and 44 positioned near the top edge 14 of the housing 12. The pin holes 42 and 44 allow for the pins or bolts described above to be inserted through the pin holes 42 and 44 and through corresponding holes on the blade assembly in order to couple the blade assembly to the housing 12. The pin holes 42 and 44 are positioned across from one another such the blade assembly is able to rotate on the pins within the housing 12. Notches 46 and 48 are also included such that the housing 12 does not impede the rotation of the blade assembly.

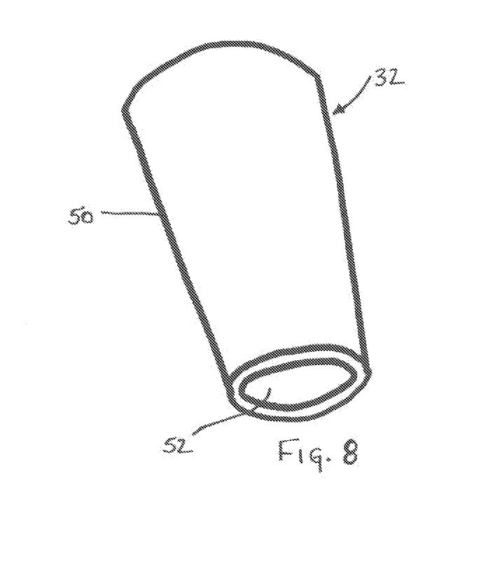

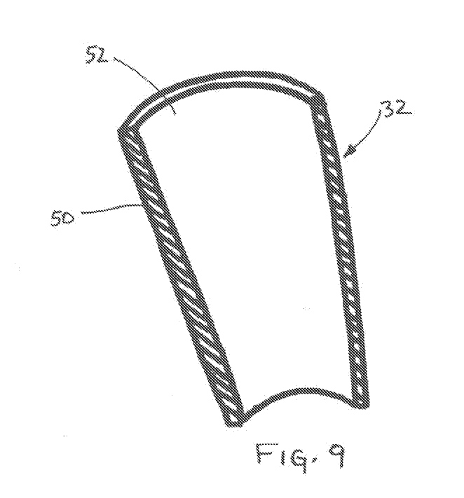

FIG. 8 illustrates a perspective view of a handle according to an embodiment of the present invention and FIG. 9 illustrates a sectional view of the handle according to the embodiment of the invention illustrated in FIG. 8. As illustrated in FIGS. 8 and 9, the handle 32 includes an outer wall 50 that defines a hollow interior space 52. The hollow interior space 52 can be positioned over an opening defined by the blade assembly or over a portion of the blade assembly made from a translucent or transparent material. In this way, the physician performing the skin biopsy is able to visualize the field of the procedure. Alternately, the handle 32 can be formed from a solid piece of material known to one of skill in the art and suitable for use as a handle for the skin biopsy device. The handle 32 can also be made with ergonomic properties or materials.

While this device has been described for use in performing a skin biopsy, it need not be limited to this application and could be used any time a sample of a predetermined depth is needed. The device can also be used for any other suitable use known to one of skill in the art. The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is desired to limit the invention to the exact construction and operation illustrated and described, and accordingly, all suitable modifications and equivalent may be resorted to, falling within the scope of the invention.

1. A device for performing a biopsy of skin comprising:
a generally curved blade configured to cut the skin at a predetermined depth;
a blade holder assembly and wherein the generally curved blade is coupled to the blade holder assembly;
a housing having an outer wall and said outer wall defining an interior space within the housing, such that the blade holder assembly and generally curved blade are disposed within the inner space of the housing; and
a handle coupled to the blade holder assembly and configured to rotate the blade holder assembly and in turn the generally curved blade within the inner space of the housing.

2. The device of claim 1 wherein the blade holder assembly comprises an outer housing defining a generally spherical outer shape and a hollow interior space.

3. The device of claim 1 wherein the housing is generally cylindrical in shape.

4. The device of claim 1 wherein a height of a position of the generally spherical blade holder within the housing correlates to a depth of a cut of the curved blade into the skin.

5. The device of claim 4 wherein the depth of the cut of the curved blade is configured to be at least 0.2 mm.

6. The device of claim 1 wherein the handle further comprises an outer wall defining a hollow interior space within the handle.

7. The device of claim 1 wherein the handle is formed from a generally clear material.

8. The device of claim 1 wherein the blade holder assembly is formed from a biocompatible metal.

9. A device for performing a biopsy of skin comprising:
a generally spherical blade assembly having an outer wall defining a hollow interior space, and wherein the outer wall further defines an opening into the hollow interior space;
a blade wherein the blade is an edge of the outer wall of the generally spherical blade assembly adjacent to the opening into the hollow interior space and wherein an edge of the blade is sharpened;
a housing having an outer wall and said outer wall defining an interior space within the housing, such that the generally spherical blade assembly is disposed substantially within the inner space of the housing; and
a handle coupled to the generally spherical blade assembly and configured to rotate the generally spherical blade assembly within the inner space of the housing.

10. The device of claim 9 wherein the opening into the hollow interior space has a wedge shape.

11. The device of claim 9 wherein the housing is generally cylindrical in shape.

12. The device of claim 9 wherein a height of a position of the generally spherical blade holder within the housing correlates to a depth of a cut of the curved blade into the skin.

13. The device of claim 12 wherein the depth of the cut of the blade is configured to be at least 0.2 mm.

14. The device of claim 9 wherein the handle further comprises an outer wall defining a hollow interior space.

15. The device of claim 9 wherein the handle is formed from a generally clear material.

16. The device of claim 9 wherein the generally spherical blade assembly is formed from a biocompatible metal or plastic.

17. A method of performing a skin biopsy comprising:
positioning a housing for a device to perform skin biopsy over an area of skin to be biopsied;
rotating a generally spherical blade assembly within the housing for the device to perform skin biopsy;
cutting the area of skin to be biopsied with a blade disposed in the generally spherical blade assembly to create a skin sample for biopsy; and
containing the skin sample for biopsy within the generally spherical blade assembly.

18. The method of claim 17 further comprising controlling a depth of a cut of the blade into the skin by adjusting to a height of a position of the generally spherical blade assembly within the housing.

19. The method of claim 18 further comprising setting the depth of the cut of the blade into the skin to at least 0.2 mm.

20. The method of claim 17 further comprising rotating the generally spherical blade assembly with a handle coupled to the generally spherical blade assembly.
